(19) United States
(12) Patent Application Publication
(10) Pub. No.: US 2007/0077274 A1
(43) Pub. Date: Apr. 5, 2007

(54) METHOD FOR PRODUCING SHAPED BODIES BASED ON CROSSLINKED GELATINE

(75) Inventor: Michael AHLERS, Eberbach (DE)

Correspondence Address: LEYDIG VOIT & MAYER, LTD, 700 THIRTEENTH ST. NW, SUITE 300, WASHINGTON, DC, 20005-3960, US

(73) Assignee: GELITA AG, Eberbach (DE)

(21) Appl. No.: 11/555,295

(22) Filed: Nov. 1, 2006

Related U.S. Application Data
(63) Continuation-in-part of application No. PCT/EP05/05174, filed on May 12, 2005.

(30) Foreign Application Priority Data
May 12, 2004 (DE) 10 2004 024 635.1

(51) Int. Cl. A61K 38/39; A61K 35/30; C08G 63/91; C08G 63/48

(52) U.S. Cl. 424/423; 424/93.7; 525/54.1

(57) ABSTRACT

A method for producing shaped bodies based on crosslinked gelatin, which may be used as carrier material for tissue implants and have an individually adjustable degradation time, is disclosed, comrising: a) preparing an aqueous gelatin solution; b) partially crosslinking the dissolved gelatin; c) producing a shaped body starting off from the gelatin solution with the partially crosslinked gelatin; and d) crosslinking the gelatin contained in the shaped body.

### CROSS REFERENCE TO RELATED APPLICATIONS

This application is a continuation-in-part of international application number PCT/EP2005/005174, filed on May 12, 2005, that claims the benefit of German patent application number 10 2004 024 635.1, filed on May 12, 2004, both of which are incorporated herein by reference in their entireties.

### BACKGROUND OF THE INVENTION

The invention relates to a method for producing shaped bodies based on crosslinked gelatin. The invention further relates to shaped bodies based on crosslinked gelatin, in particular, sheet materials and hollow bodies. The invention also relates to implants which are manufactured using the aforementioned shaped bodies.

So-called tissue implants, which are constructs consisting of a carrier material and living cells (tissue engineering), may be used for treating damaged tissues and organs. Such implants are known in the prior art and are used, inter alia, for the regeneration of skin or cartilage.

The carrier material has to be of such a kind that it will promote the growth and proliferation of the cells. In addition, a certain firmness is desirable in order to protect the cells against mechanical stress during growth in the body. At the same time, the material should, however, be flexible enough to adapt to the shape of the body part to be treated. Finally, the carrier material should be capable of being resorbed as completely as possible by the body after the cells have grown to a sufficient extent and have synthesized an extracellular matrix.

The materials used to date are unable to meet these manifold requirements to the desired extent. Inter alia, carriers based on chitosan, alginate, agarose and hyaluronic acid are described in the prior art. The three last-mentioned materials partly exhibit considerable deficiencies in terms of residue-free resorption.

Another carrier material that is often used is collagen. However, this is not obtainable in a composition and purity that can be reproduced in the desired manner. Furthermore, the collagen obtained from animal sources may contain immunogenic telopeptides which can trigger rejection by the body.

Moreover, all of the aforementioned materials have the disadvantage that the respective resorption time after which the material has disintegrated is not individually adjustable. The optimum length of time may differ depending on the type of tissue to be treated and the size of the defect. For example, owing to the slow growth of the chondrocytes, degradation times of four weeks and more are desirable for the regeneration of cartilage defects.

The object underlying the present invention is, therefore, to provide a method for obtaining materials which meet the requirements described hereinabove and with which, in addition, the respective degradation time of the material can be adjusted in a specific manner.

This object is accomplished with the method mentioned at the outset, in accordance with the invention, in that it comprises the following steps:

a) preparing an aqueous gelatin solution;

b) partially crosslinking the dissolved gelatin;

c) producing a shaped body starting off from the gelatin solution with the partially crosslinked gelatin; and

d) crosslinking the gelatin contained in the shaped body,

The use of crosslinked gelatin as starting material for wound dressings and tissue implants has already been described as such in the prior art. In contrast to collagen, gelatin is a product with a defined composition, which can also be produced with a very high degree of purity. In addition, materials made from gelatin are optically clear, whereas products made of collagen mostly have a milky, cloudy appearance. The latter may prove disadvantageous in a light-microscopical analysis of the cell growth.

However, the crosslinked gelatin materials known to date do not exhibit a stability that is required for long-term applications. For example, a crosslinking with 1,5-pentanedial of up to 12 hours, as described in European patent EP 1 053 757, is not sufficient to obtain a gelatin material suitable for the regeneration of cartilage defects. Nor are sponges made of crosslinked gelatin, as already in use for treating wounds and bleeding, suitable, as they are partly disintegrated within a few minutes in the presence of proteases.

It has been found that a higher degree of crosslinking of the gelatin involves an increased stability.

An increase in the stability by means of a higher concentration of crosslinking agents or longer duration of the crosslinking reaction is limited by a gelatin solution no longer being able to be processed and shaped when the crosslinking is too high.

A crosslinking of the gelatin solely after the production of the shaped body does not produce satisfactory results either, as a higher degree of crosslinking of the gelatin occurs at the interfaces accessible from the outside than in the inner areas of the shaped body. For example, in the case of shaped bodies with a cell structure, to which reference will be made in detail hereinbelow, this may result in the cell walls or webs between the pores in the interior being only insufficiently crosslinked and disintegrating too rapidly during the later use of the shaped bodies.

### BRIEF SUMMARY OF THE INVENTION

Surprisingly, the method according to the invention described hereinabove, which is characterized by a two-step crosslinking of the gelatin materials, not only makes it possible to produce materials which have a correspondingly long life and are stable in terms of shape, without having to renounce the advantages of gelatin described hereinabove. The method also enables the desired resorption time of the material to be individually adjusted.

The shaped bodies produced by the method according to the invention are thereby self-supporting, i.e., they are sufficiently stable for handling and use without a carrier element. This is highly advantageous in medical use, as materials that are as uniform as possible have to be used here.

### DETAILED DESCRIPTION OF THE INVENTION

Gelatin of different origin and quality may be used as starting material for the method. Depending on the embodiment of the invention, the gelatin concentration in the solution (a) may be from 5 to 45 wt %, preferably from 10 to 30 wt %.

The shaped body (c) formed after the first crosslinking (b) is preferably at least partially dried before the second crosslining (d), preferably up to a residual moisture content of less than 20 wt %, in particular, 15 wt % or less.

The second crosslinking may be carried out by the action of an aqueous solution of a crosslinking agent, but the action of a gaseous crosslinking agent is preferred.

In principle, all compounds which bring about a chemical crosslinking of the gelatin may be used as crosslinking agent. Aldehydes, dialdehydes, isocyanates, diisocyanates, carbodiimides and alkyl dihalides are preferred, and the same or different compounds may be used for the two crosslinking steps.

Particularly preferred is the use of formaldehyde, especially for the second crosslinking step in the gaseous phase, as the shaped body can be simultaneously sterilized by the formaldehyde. The action of the formaldehyde on the shaped body can be promoted by a water vapor atmosphere.

The properties of the shaped bodies produced in accordance with the method according to the invention can be further improved in terms of their stability by the shaped bodies being subjected to a subsequent heat treatment at reduced pressure after the second crosslinking step. This subsequent treatment is preferably carried out at temperatures of from 80 to 160° C., as the observed effects are relatively indistinct below 80° C., and an undesired discoloring of the gelatin may occur above 160° C. Values ranging from 90 to 120° C. are most preferred.

Reduced pressure is to be understood as pressures below atmospheric pressure, with pressure values that are as low as possible, in the ideal case a vacuum, being preferred.

The subsequent heat treatment has an advantageous effect in two different respects. Firstly, under the above-mentioned temperature and pressure conditions, a further, dehydrothermal crosslinking of the gelatin takes place by different amino acid side chains reacting with one another and water thereby being eliminated. This is promoted by the eliminated water being removed from the equilibrium by the low pressure. Therefore, owing to the subsequent heat treatment, given the same amount of crosslinking agents, a higher degree of crosslinking can be achieved, or, given a comparable degree of crosslinking, the amount of crosslinking agents can be reduced.

The further advantage of the subsequent heat treatment is that the residual content of crosslinking agent that is not used up and remains in the shaped body can be significantly reduced.

To ensure good biocompatibility of the shaped bodies, for example, when used as carrier material for tissue implants, excess crosslinking agent which has not reacted is preferably removed from the shaped body in the method according to the invention. This may be done, for example, by degassing the shaped bodies at normal pressure for several days and/or by washing with a liquid medium, with the latter also requiring a length of time of from one day to one week, depending on the concentration of the crosslinking agent, size of the shaped body, etc.

Since, owing to the subsequent heat treatment described hereinabove, on the one hand, the amount of crosslinking agent that is used can be reduced, and, in addition, excess crosslinking agent can be removed from the shaped body by the elevated temperature and the reduced pressure, a significant reduction in the residual content of crosslinking agent can already be achieved within approximately 4 to 10 hours by this additional method step.

Therefore, shaped bodies according to the invention, which are preferably substantially free from excess crosslinking agent, can be produced with a relatively low expenditure of time owing to the subsequent heat treatment.

The shaped bodies according to the invention preferably have a content of crosslinking agent of approximately 0.2 wt % or less, which, for example, in the case of the crosslinking agent formaldehyde represents a limit value for the biocompatibility of carrier materials. This value cannot be attained in the above-mentioned length of time of 4 of 10 hours by pure washing with liquid medium.

Surprisingly, a heat treatment at reduced pressure does, in fact, only result in improved stability of the shaped bodies according to the invention when this, as described hereinabove, is carried out after the two crosslinking steps. A prior treatment of the gelatin used under the corresponding temperature and pressure conditions does not result in any appreciable increase in the lifetime of the shaped bodies, although the gelatin is chemically modified in this case, too, which is reflected in an increase in the Bloom strength, the viscosity and the average molecular weight.

A prior heat treatment of the gelatin used, which is preferably carried out under comparable conditions to those of the subsequent heat treatment of the shaped bodies, does, however, bring about other advantages, which, depending on the application, may be of significance. Firstly, the prior heat treatment results in a greater tear strength of the shaped bodies according to the invention in the dry state, particularly in the case of the films described hereinbelow. Furthermore, owing to the higher viscosity of the gelatin having undergone prior heat treatment, the concentration of the gelatin solution to be used can be reduced, whereby shaped bodies with a lower density and greater flexibility are obtainable. This applies primarily to shaped bodies with a cell structure, which will be described in detail hereinbelow.

A gelatin with a viscosity of 8 mPas or more is preferably used for the method according to the invention. This value relates to the viscosity of a 6.7 wt % aqueous gelatin solution at 60° C.

The desired strength, in particular, tear strength, and stability, and the lifetime or degradation behavior of the material produced can be adjusted very easily with the method according to the invention, preferably by the specific choice of the conditions under which it is produced. For example, both strength and lifetime can, as a rule, be increased by a higher concentration of the crosslinking agent or by die subsequent heat treatment described hereinabove.

Thus, shaped bodies can, surprisingly, be obtained, which, on the one hand, under physiological conditions, specifically remain stable, for example, for longer than one week, longer than two weeks or longer than four weeks, and, on the other hand, meet the requirements with respect to cell compatibility and resorbability.

The term stability in this context is to be understood as the material substantially maintaining its original shape both during storage in the dry state and during the stated period of time under standard physiological conditions and only subsequently being resorbed to a considerable extent.

Standard physiological conditions to which the material is subjected when used for producing implants are, primarily, characterized by temperature, pH value and ionic strength. Corresponding conditions can be defined in vitro by an incubation of the material in PBS buffer (pH 7.2, 0.09 wt % NaCl) at 37° C. in order to test and compare various materials in terms of their time-dependent stability behavior.

Also the resistance of the shaped bodies to proteases, which are mainly responsible for the degradation of the material, can already be estimated very well in vitro by adding a protease, for example, pepsin, or from colonization with protease-producing cells, for example, fibroblasts. Quantitative details thereof may be taken from the embodiments given hereinbelow.

In spite of the to some extent very high degree of crosslinking achievable with the described method, the shaped bodies produced nevertheless have sufficient flexibility to meet the requirements for use as tissue implant such as, for example, pliability and suturability.

The desired flexibility can preferably be adjusted by adding softeners in the course of the production method. As a rule, an increase in the softener concentration will result in more flexible shaped bodies. Glycerin, oligoglycerins, oligoglycols and sorbite are, for example, suitable as softener.

Various methods may be used for producing the shaped bodies from the crosslinked gelatin solution, for example, molding or extruding, optionally combined with foaming, if cellular materials are aimed at.

The present invention further relates to shaped bodies made from crosslinked gelatin, wherein the degree of crosslinking is selected such that under physiological conditions the shaped bodies will remain stable for a predetermined time, for example, at least one, two or four weeks. A preferred method for producing such shaped bodies is that which is described hereinabove.

In a preferred embodiment, the shaped bodies are sheet materials. Sheet materials may be used in many applications as carrier for tissue implants, for example, in the regeneration of skin. The sheet material may be cellular, i.e., have a cell structure, or be in the form of (non-cellular) film.

Cell structures such as, for example, sponges or foams, can be obtained by foaming the gelatin solution with a gas, in particular, air. Preferred cell structures are open-pored so as to enable ingrowth of cells and formation of a three-dimensional tissue structure when used for tissue implants.

The density of the shaped bodies with a cell structure and the pore width may be adjusted within a wide range, preferably by the intensity of the foaming. Moreover, the density may be lowered by using a gelatin that has undergone prior heat treatment or a gelatin with a high viscosity, as described hereinabove.

The properties of a shaped body with a cell structure according to the invention may also be influenced by the cell structure being modified by mechanical action on the shaped body. Mechanical action includes, for example, pressing or rolling the shaped body to such an extent that some of the cell walls or webs between the pores of the cell structure are broken.

The density of the shaped body is preferably increased by a factor of from 2 to 10 by the mechanical action.

The flexibility of the shaped bodies in the dry state can be increased by the mechanical action without the stability behavior with respect to time being noticeably influenced. This is advantageous, as particularly flexible sheet materials, when used as tissue implant, can adapt better to the conditions of the body.

The pores of the cell structure preferably have an average diameter of less than 300 μm. In the case of larger average pore diameters, too low a retaining capability is often observed when cells are introduced into the cell structure, The preferred lower limit of the pore width complies, in most cases, with the size of the cells that are used and are to grow in all three dimensions into the cell structure.

A gelatin solution with a concentration of from 5 to 25 wt %, preferably from 10 to 20 wt %, can be used for producing shaped bodies with a cell structure. In general, a higher gelatin concentration results in a higher breaking strength of the shaped bodies. Surprisingly, this works substantially independently of the degree of crosslinking, via which the lifetime of the material can be adjusted.

Preferred shaped bodies with a cell structure are reversibly compressible. This applies, in particular, in the hydrated state, with the extent of the compressibility being dependent, inter alia, on the gelatin concentration used and the pore width.

The term “films” is to be understood as thin sheet materials without a cell structure. They can be produced by molding from a preferably substantially degassed gelatin solution.

A preferred embodiment relates to flexible films, the flexibility of which can be adjusted by, for example, adding softeners. The compounds already described in conjunction with the method according to the invention can be used as softeners. Under standard physiological conditions, the stability of the films remains substantially uninfluenced by the use of the softeners.

Films with a thickness of from 20 to 500 μm are preferred, most preferred from 50 to 100 μm.

Preferably gelatin solutions with a concentration of from 5 to 45 wt % further preferred approximately from 10 to 30 wt %, are used for producing the films.

A further preferred embodiment of the invention relates to a multi-layered material comprising a film and a sheet material with a cell structure. The two layers can be directly joined to each other, which can be brought about by, for example, the sheet material with a cell structure being brought into contact with the film, optionally pressed into it, before the film is dried.

Alternatively, the layers can be adhesively joined to each other, and an adhesive based on gelatin will preferably be used as adhesive.

In the case of the multi-layered sheet material according to the invention, the film and the sheet material with a cell structure will preferably be joined with surface-to-surface contact, in particular, over the entire surface, to each other.

In another preferred embodiment, the shaped bodies may also be in the form of a hollow body, in particular, a hollow section. Such hollow sections are, for example, obtainable by extrusion of the gelatin solution. Alternatively, hollow sections with a cell structure described hereinabove can be produced by simultaneous extruding and foaming.

However, hollow sections can also be formed from previously produced sheet materials, in particular, films, for example, by rolling-up.

A preferred embodiment relates to cylindrical hollow sections, for example, small tubes. These, too, can be produced, inter alia, by rolling up the sheet materials described hereinabove.

In addition to the materials described hereinabove, the shaped bodies according to the invention may also have any other shape or structure. In particular, shaped bodies that are spatially adapted to the tissue defect to be treated can be used as tissue implant.

The invention further relates to the use of the described shaped bodies for use in the field of human and veterinary medicine and for the manufacture of implants.

One use according to the invention relates to the manufacture of dressings for wounds made from the materials described hereinabove. These can be used in the treatment of wounds or internal or external bleeding, for example, during operations. The resorption of the material occurs after an individually adjustable time, preferably determined by the manufacturing condition that is selected.

It has been found that the shaped bodies according to the invention are extremely well suited for colonization with mammalian cells, i.e., with human or animal cells, A shaped body can be treated with a suitable nutrient medium, and the cells, for example, fibroblasts or chondrocytes, subsequently sown thereon. Owing to the stability of the material, the cells can grow and proliferate in vitro for several weeks.

The invention further relates to implants, in particular, tissue implants, comprising a shaped body according to the invention and cells cultivated thereon, as described hereinabove.

The implants according to the invention are used for treating tissue defects, for example, skin or cartilage defects, and the sown-out cells may, for example, be previously taken from the patient. During the growth phase of the cells, the shaped body protects the tissue from mechanical stress while it is forming, and formation of the extracellular matrix by the cells is made possible. The resorption time adjustable in accordance with the invention proves to be of particular advantage. With the use of long-lasting materials according to the invention, which have a resorption time of more than four weeks, also defects covering large areas or defects in types of tissue with slow cell growth may be treated.

Shaped bodies with a cell structure are particularly preferred for use in implants, as a three-dimensional tissue structure can develop here by the cells growing into the shaped body. Owing to a reversible compression of the shaped body, a cell suspension can be absorbed and the cells homogeneously distributed in the shaped body.

Depending on the field of application, a sheet material with a cell structure can be used, for example, for treating damage, extensive injuries to, or burns on, the skin. However, any other shape may also be advantageous, for example, individual, three-dimensional shaped bodies for treating cartilage defects, e.g., damage and/or injuries to the cartilage tissue.

In a further preferred embodiment of the invention, the implant comprises a multi-layered sheet material described hereinabove. In such an implant, the sheet material with a cell structure serves as carrier for the cells, while the film offers additional mechanical protection.

Such a construct may be advantageous, for example, for the regeneration of cartilage tissue that grows very slowly.

The invention further relates to nerve guidance channels. The implantation of nerve guidance channels serves for the regeneration of severed nerve cords. The channel should be of such dimensions that an individual nerve cell can grow therein. This is ensured with a preferred inner diameter of 1 mm. In addition, the nerve guidance channel should be of such a nature that blood vessels can penetrate it from the sides so as to enable the nerve cell to be supplied with nutrients.

Nerve guidance channels which meet this requirement can be produced with the method according to the invention.

In a preferred embodiment, the nerve guidance channel is produced by rolling up a sheet material according to the invention described hereinabove, in particular, a film.

### BRIEF DESCRIPTION OF THE SEVERAL VIEWS OF THE DRAWINGS

These and further advantages of the invention will be explained in greater detail by the examples and figures given hereinbelow, There are shown in:

FIG. 1: a stress-strain diagram of films according to the invention;

FIG. 2: a stress-strain diagram of further films according to the invention;

FIG. 3: degradation behavior with respect to time of shaped bodies with a cell structure according to the invention;

FIG. 4: photomicrographs of shaped bodies with a cell structure according to the invention;

FIG. 5: a breaking strength diagram of shaped bodies with a cell structure according to the invention;

FIG. 6: protease-resistance of shaped bodies with a cell structure according to the invention;

FIG. 7: degradation behavior with respect to time of further shaped bodies with a cell structure according to the invention;

FIG. 8: a stress-strain diagram of further films according to the invention;

FIG. 9: cell distribution of chondrocytes in shaped bodies according to the invention; and

FIG. 10: a photographic representation of the colonization of a film according to the invention with fibroblasts.

### EXAMPLES

### Example 1

### Production and Properties of Films Based on Crosslinked Gelatin

Pig skin gelatin (Bloom strength 300) was dissolved in four different batches in a mixture of water and glycerin in accordance with the amounts stated in Table 1 at 60° C. Following degassing of the solutions ultrasonically, the amount indicated in Table 1 of an aqueous formaldehyde solution (1.0 wt %, room temperature) was added, the mixture homogenized and applied at approximately 60° C. in a thickness of 1 mm with a doctor blade to a polyethylene base.

TABLE 1

| | Batch | | | | | | | |
|---|---|---|---|---|---|---|---|---|
| | 1-1 | | 1-2 | | 1-3 | | 1-4 | |
| Gelatin | 30 | g | 30 | g | 30 | g | 30 | g |
| Water | 56 | g | 51 | g | 45 | g | 40 | g |
| Glycerin | 14 | g | 14 | g | 20 | g | 20 | g |
| Formaldehyde | — | | 10 | g | 5 | g | 10 | g |
| solution | | | | | | | | |
| Formaldehyde | 0 | | 3333 | ppm | 1667 | ppm | 3333 | ppm |
| content in relation | | | | | | | | |
| to the gelatin | | | | | | | | |

After drying at 30° C. and a relative air humidity of 50% for about one day, the films were removed from the PE base and dried again for approximately 12 h under the same conditions.

The dried films had a thickness of less than 100 μm and for the second crosslinking step were subjected for two hours to the equilibrium vapor pressure of a 17% aqueous formaldehyde solution at room temperature in a desiccator. In the case of the film produced in accordance with batch 1-1, the second crosslinking step was the only one.

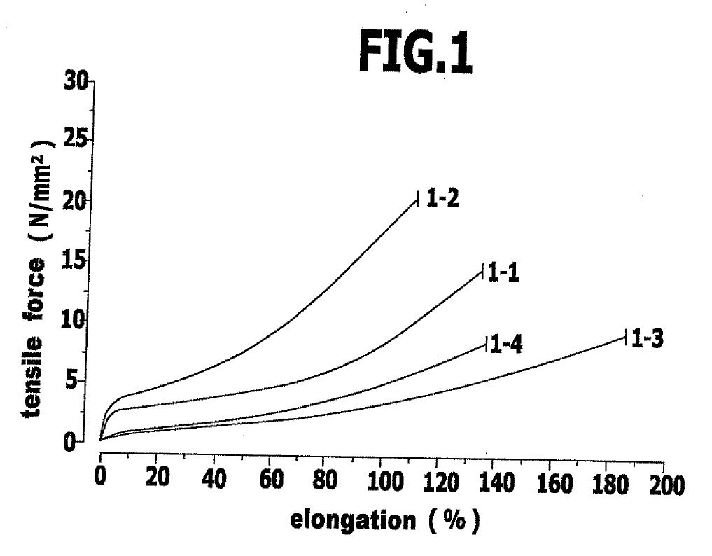

The mechanical properties of the various films (in the dry state) are shown in FIG. 1: whereas, in comparison with film 1-1, film 1-2 exhibits, owing to the two-step crosslinking, a higher tear strength with less elongation at breaking point, film 1-3 is considerably more expansible (flexible) owing to the increase in the glycerin concentration. Owing to the higher crosslinking agent concentration in film 1-4 in comparison with film 1-3, a somewhat higher strength with less elongation at breaking point can, in turn, be obtained.

Films in accordance with batches 1-1 and 1-2 were also produced. However, these were not subsequently subjected to any crosslinking in the gaseous phase (films 1-1′, uncrosslinked and 1-2′, crosslinked once). The elongation at breaking point curves of these films end at approximately 140% / 10 N/mm² (1-1′) and 115% / 15 N/mm² (1-2′), respectively, and, for reasons of clarity, are not shown in FIG. 1.

It will be understood that the respective curve forms relating to production on a laboratory scale cannot be reproduced exactly. The relationship of the curves of various films to one another is, however, characteristic.

Accordingly, the example shows that with the method according to the invention, the flexibility of the films produced can be adapted over a wide range by both the degree of crosslinking and the amount of softener being varied accordingly.

The films that are crosslinked twice are distinguished by their remaining stable for a considerably longer time under standard physiological conditions:

The degradation behavior of the films was measured by placing pieces of film measuring 2×3 cm into 500 ml PBS buffer (pH 7.2, 0.09 wt % NaCl) in each case and the concentration of the gelatin dissolved in the buffer was photometrically determined at a wavelength of 214 nm. Whereas the films that had not been crosslinked or crosslinked once were already completely disintegrated after 15 minutes, in the films that had been crosslinked twice there was still no change to be observed after one hour.

### Example 2

### Production and Properties of Films Based on Crosslinked Gelatin

Eight batches of a 30 wt % solution of pig skin gelatin (Bloom strength 300) in water/glycerin in accordance with the amounts stated in Table 2 were produced by dissolving the gelatin at 60° C. Following degassing of the solutions ultrasonically, the corresponding amounts of an aqueous formaldehyde solution (1.0 wt %, room temperature) were added, so that the final concentration of formaldehyde corresponded respectively to the value given in Table 2. As described, for the rest, in Example 1, films were produced from the mixtures, dried and, in the given case, crosslinked (cf. Table 2).

TABLE 2

| | Formaldehyde in the | | Duration of | Glycerin (in |
|---|---|---|---|---|
| | first crosslinking | | the second | relation to |
| Batch | (in relation to gelatin) | | crosslinking | gelatin) |
| 2-1 | 0 | | none | 47% |
| 2-2 | 600 | ppm | none | 47% |
| 2-3 | 600 | ppm | 2 h | 47% |
| 2-4 | 4800 | ppm | none | 47% |
| 2-5 | 4800 | ppm | 2 h | 47% |
| 2-6 | 4800 | ppm | 17 h | 47% |
| 2-7 | 4800 | ppm | 2 h | 23% |
| 2-8 | 4800 | ppm | 17 h | 23% |

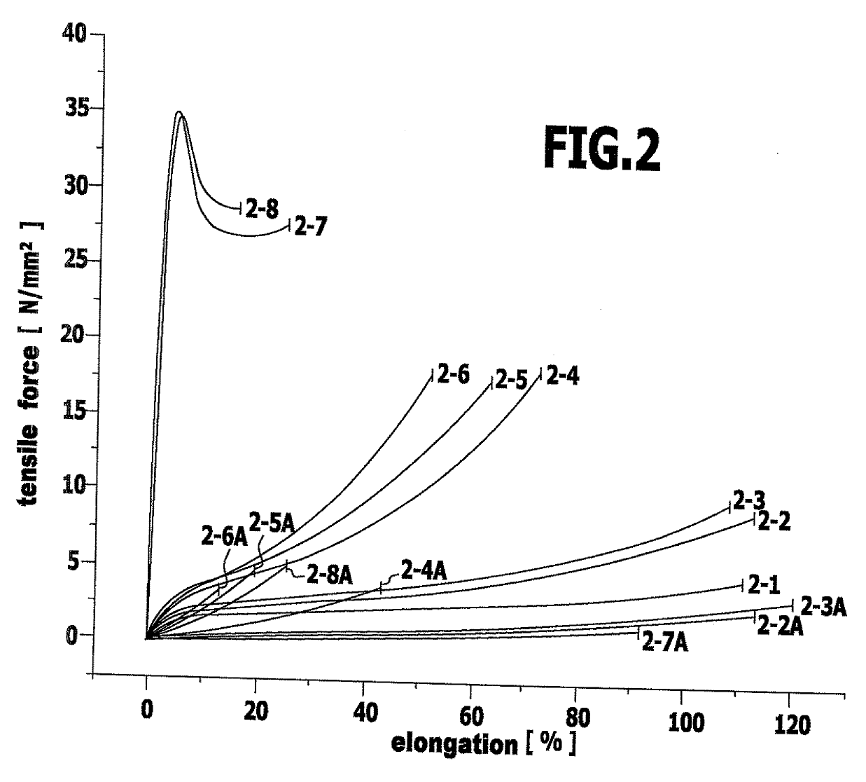

The stress-strain properties of the eight films are shown in FIG. 2.

Curves 2-1 to 2-8 relate to the corresponding dry films, curves 2-2A to 2-8A to the hydrated films which were placed for four hours in PBS buffer (the uncrosslinked film 2-1 disintegrates under these conditions to such an extent that examination of the stress-strain properties is not possible). The vertical marks indicate the end points of the respective curves.

It is also clear from this example that the tear strength and flexibility, respectively, of the films can be varied over a wide range by means of the different production conditions.

Furthermore, it is evident that also films which in the dry state are relatively stiff (which may be advantageous for the processing), after their hydration, may, in some cases, become very flexible under physiological conditions:

Films 2-7 and 2-8, which in the dry state exhibit almost identical properties, produce after their hydration, in the one case, an extremely flexible material (2-7A), as required, for example, for use in the joint area, in the other case, a stiffer material with a higher tear strength (2-8A), which may be used, for example, in the bone area.

### Example 3

### Production and Properties of Shaped Bodies with a Cell Structure Based on Crosslinked Gelatin

Five batches of a 12 wt % solution of pig skin gelatin (Bloom strength 300) were produced in water by dissolving the gelatin at 60° C., degassed ultrasonically, and the corresponding amount of an aqueous formaldehyde solution (1.0 wt %, room temperature) was added respectively, which resulted in 1500 ppm formaldehyde (in relation to the gelatin). No formaldehyde was added to a correspondingly produced reference sample.

The homogenized mixtures were tempered after a reaction time of 10 minutes to 45° C. and mechanically foamed with air. The foaming procedure lasting approximately 30 minutes was carried out with a different relationship of air to gelatin solution for the five batches, whereby cell structures with different wet densities and pore sizes in accordance with Table 3 were obtained.

The foamed gelatin solutions, which exhibited a temperature of 26.5° C., were cast in molds measuring 40×20×6 cm and dried for approximately four days at 26° C. and a relative air humidity of 10%.

The dried shaped bodies with a sponge-like cell structure (referred to hereinbelow as sponges) were cut into layers of 2 mm thickness and for the second crosslinking step were subjected for 17 hours to the equilibrium vapor pressure of a 17% aqueous formaldehyde solution at room temperature in a desiccator. To achieve a uniform degassing of the entire volume of the shaped bodies, the desiccator was respectively evacuated two to three times and aerated again.

The pore structure of the sponges was determined by light microscopy and was able to be confirmed by scanning electron microscopy.

TABLE 3

| | Wet density | Dry density | Average pore |
|---|---|---|---|
| Batch | (mg/cm³) | (mg/cm³) | size (μm) |
| 3-1 | 100 | 20 | 250 |
| 3-2 | 175 | 27 | 200 |
| 3-3 | 300 | 50 | 125 |
| 3-4 | 530 | 70 | 100 |
| 3-5 | 600 | 100 | 75 |
| Reference | 78 | 12 | 300 |
| (crosslinked once) | | | |

To determine the stability of the sponges, pieces measuring 30×30×2 mm were weighed, respectively placed in 75 ml PBS buffer and stored at 37° C. After the respective storage time, the pieces were washed for 30 minutes in water, dried and weighed out.

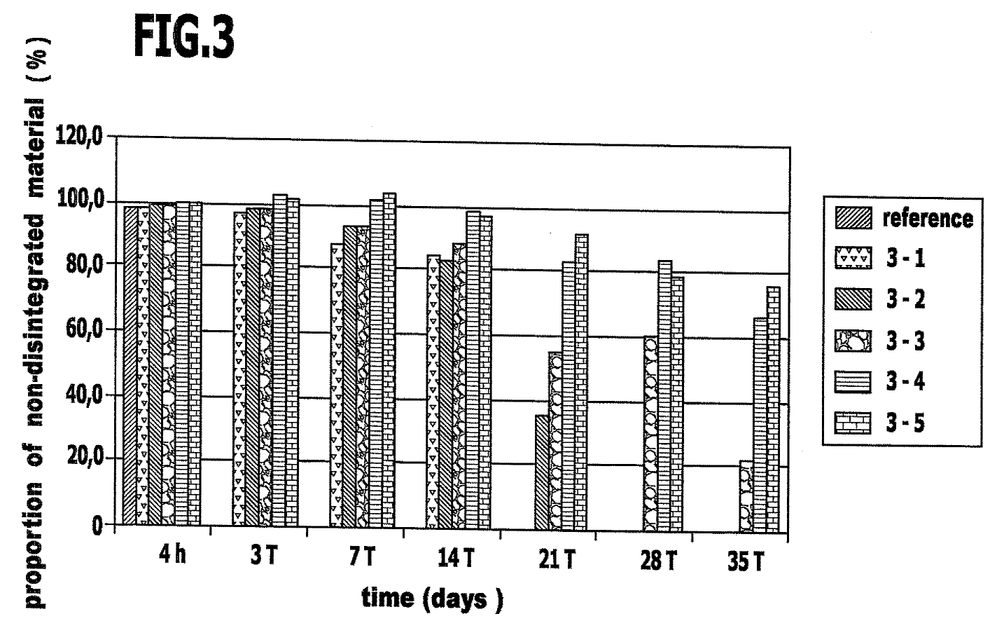

FIG. 3 shows the disintegration characteristics of the sponges 3-1 to 3-5 and of the reference sample crosslinked once (the sequence of the bars shown is respectively: reference, 3-1, 3-2, 3-3, 3-4, 3-5).

Whereas the reference sample is already completely disintegrated after three days, all of the sponges produced in accordance with the invention are still maintained to over 80% even after 14 days. Considerable differences are, however, to be found in the further degradation behavior, which are due to the different foam densities of the materials. Sponge 3-1 is completely disintegrated after 21 days and sponge 3-2 after 28, whereas sponges 3-4 and 3-5 are still substantially maintained even after 35 days. This provides the possibility of specifically influencing the degradation behavior of the cell structure materials independently of other parameters.

However, the properties of the cell structure materials can also be significantly modified by a change in the gelatin concentration in the starting solution.

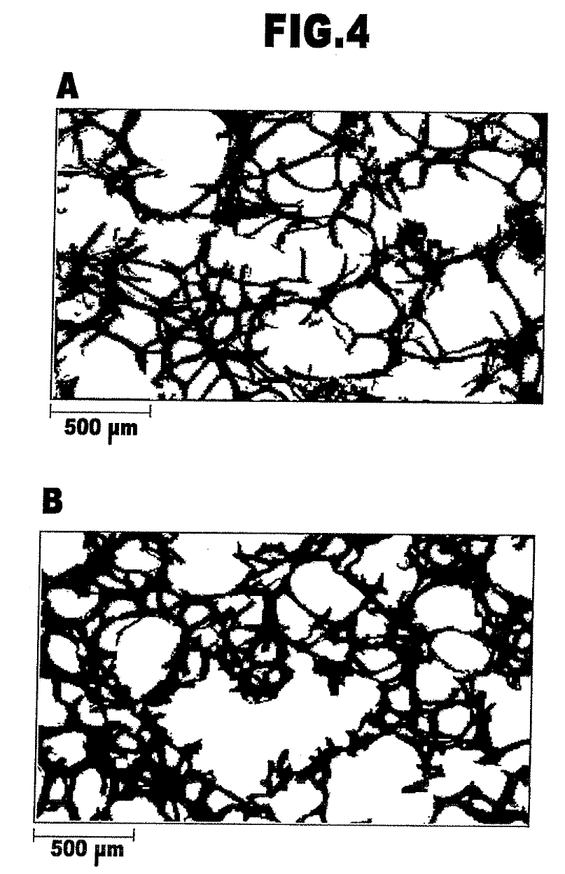

FIG. 4 shows photomicrographs of the cell structure of two shaped bodies in thin sections of 150 μm, which, starting off from a 12 wt % (illustration A) and an 18 wt % (illustration B) gelatin solution, were produced under otherwise identical conditions. The higher gelatin concentration results in wider (thicker) cell walls or webs between the individual pores, which is reflected in an increased breaking strength of the corresponding sponges.

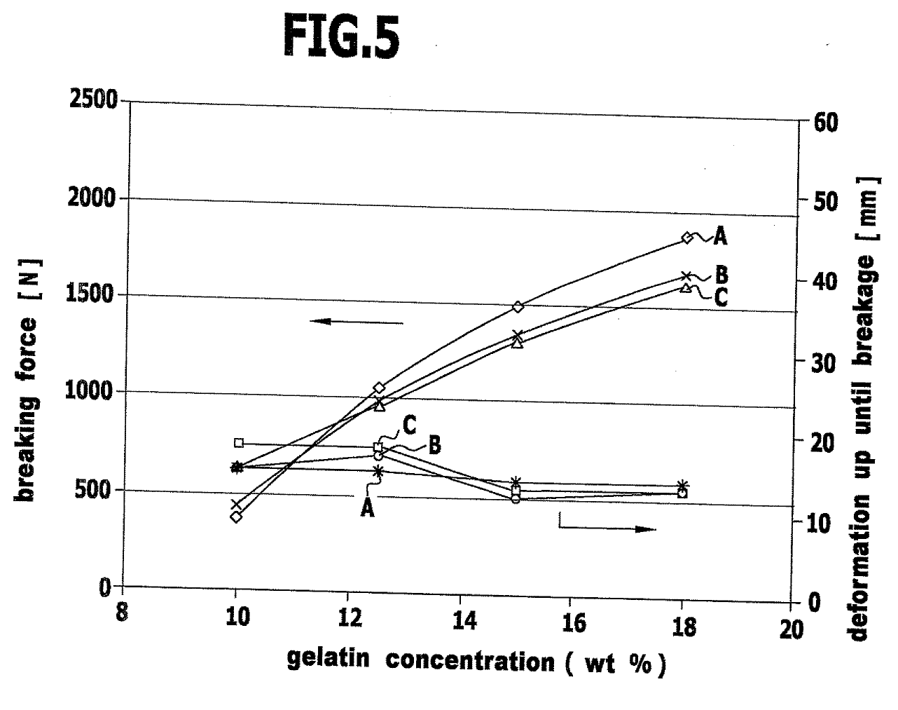

This is illustrated quantitatively in FIG. 5. The three curves A, B and C respectively represent sponges with three different degrees of crosslinking.

The breaking strength increases constantly with an increase in the gelatin concentration of the starting solution from 10 to 18 wt %, with a wide range of from approximately 500 to almost 2000 newtons being covered. At the same time, the deformation up until breakage changes only slightly. Surpisingly, the correlation between breaking force and gelatin concentration is substantially independent of the degree of crosslinking.

By means of the degree of crosslinking, i.e., by the choice of the concentration of the crosslinking agent, the stability of the shaped bodies can, on the other hand, be influenced, in particular, in view of proteolytic degradation.

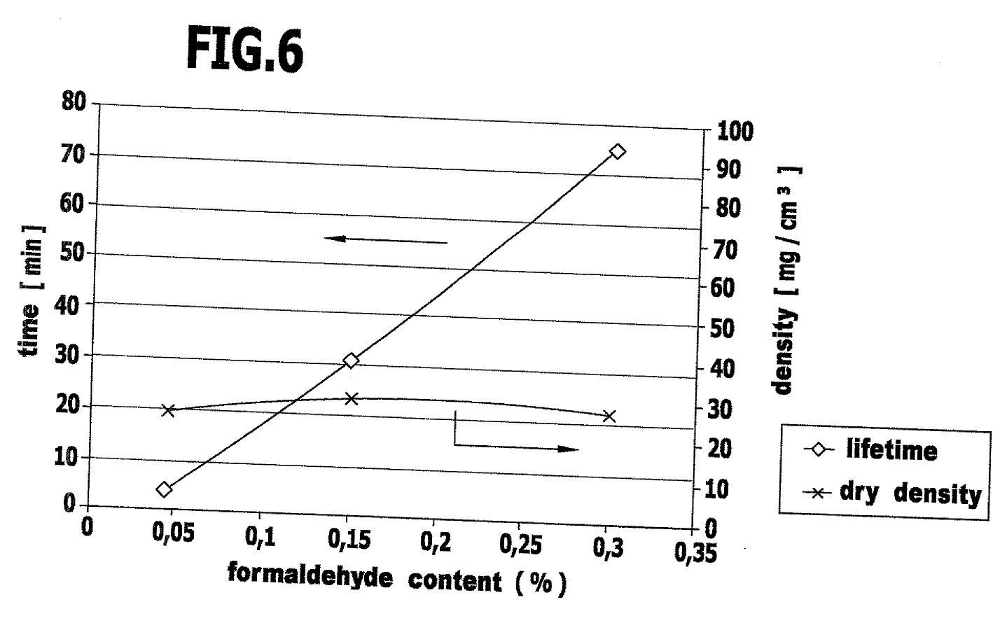

The resistance of various cell structure materials (sponges) to pepsin in dependence upon the amount of formaldehyde (wt % in relation to gelatin) used in the first crosslinking step is shown in FIG. 6.

The degradation was carried out at 37° C. in a 1.0 wt % pepsin solution in PBS buffer, the pH value of which was adjusted with HC1 to 1. With an increase in the formaldehyde concentration from 500 to 1500 up to 3000 ppm, the degradation time of the sponges rises from less than 5 min to 30 min up to 75 min. The dry density of the materials is substantially independent of the degree of crosslinking here. The very drastic degradation conditions chosen here are not to be compared with the considerably milder physiological conditions, so that considerably longer degradation times will apply under the latter conditions.

### Example 4

### Production and Properties of Shaped Bodies with a Cell Structure Based on Crosslinked Gelatin with Subsequent Heat Treatment

A 12 wt % solution of pig skin gelatin (Bloom strength 300) was produced as in Example 3, degassed ultrasonically, and mixed with the corresponding amount of an aqueous formaldehyde solution (1.0 wt %, room temperature), so that 1500 ppm formaldehyde (in relation to the gelatin) was obtained.

The homogenized mixture was tempered after a reaction time of 5 minutes to 45° C. and mechanically foamed with air for approximately 30 minutes. The foamed gelatin solution was cast in molds and dried as described in Example 3, and a shaped body with a sponge-like cell structure (sponge) with a wet density of 121 mg/cm³, a dry density of 18 mg/cm³ and an average pore size of 250 μm was obtained.

The shaped body was already firm after the first crosslinking step.

Four samples measuring 30×30×2 mm were cut from the sponge and each subjected to a second crosslinking step in the gaseous phase at an equilibrium vapor pressure of a 10% aqueous formaldehyde solution as in the method described in Example 3. However, differently from Example 3, the time during which the formaldehyde acted was significantly shorter in this case, namely 2 hours in the case of samples 4-1 and 4-3 and 5 hours in the case of samples 4-2 and 4-4.

All four samples were vacuum degassed after the second crosslinking step, and samples 4-3 and 4-4 were then subjected to a subsequent heat treatment. By means of a rotary evaporator, the sponges in question were kept for 6 hours at a vacuum of approximately 14 mbar at 105° C.

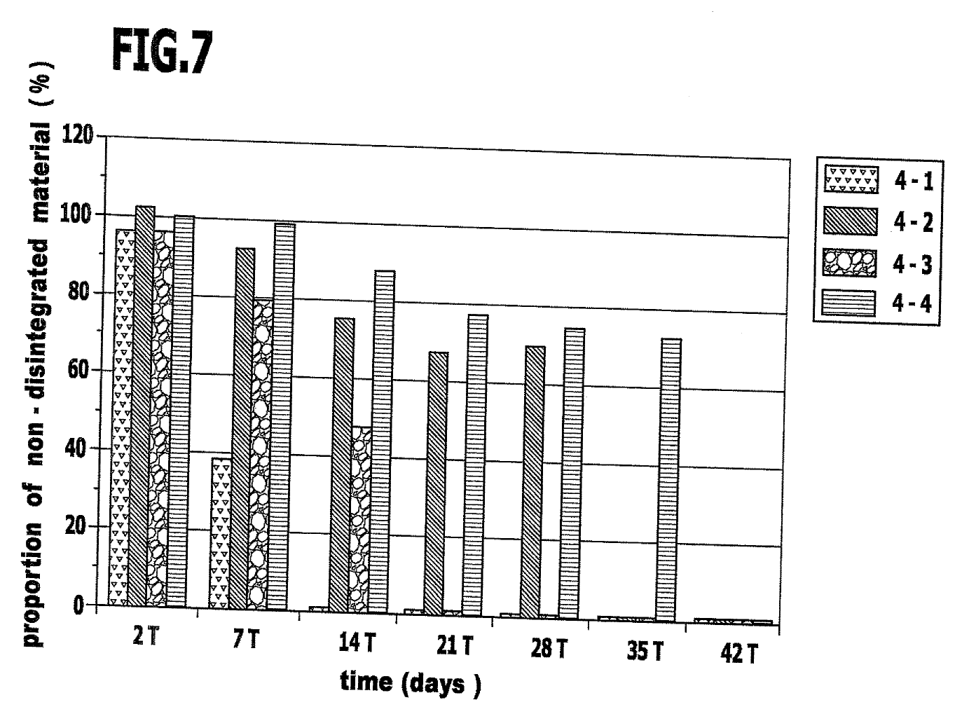

The stability of the various samples in PBS buffer was determined as described in Example 3. FIG. 7 shows the disintegration behavior of samples 4-1 to 4-4 (the sequence of the illustrated bars is respectively: 4-1, 4-2, 4-3, 4-4).

It becomes apparent that the lifetime of the sponges under physiological conditions can be significantly prolonged by the subsequent heat treatment. Whereas sample 4-1 which was not subsequently treated is already completely disintegrated after 14 days, sample 4-3 which was subsequently heat treated is still maintained to almost 50% at this point in time. A corresponding difference is also to be seen between samples 4-2 (not subsequently treated) and 4-4 (subsequently heat treated), which were each crosslinked for 5 hours in the gaseous phase. After 35 days, sample 4-2 is completely disintegrated and sample 4-4 still maintained to more than 70%.

Aside from the increase in the degree of crosslinking and the stability of the sponges, the subsequent heat treatment under vacuum has the further advantage that the residual amount of crosslinking agent remaining in the shaped body can be effectively reduced, whereby washing for a long time prior to use can be avoided or at least shortened. This applies particularly to mechanically relatively firm sponges which were produced on the basis of high gelatin concentrations.

In order to measure this effect, two batches of an 18 wt % solution of pig skin gelatin (Bloom strength 300) in water were produced by dissolving the gelatin at 60° C., degassed ultrasonically, and each mixed with the corresponding amount of an aqueous formaldehyde solution (1.0 wt %, room temperature), so as to produce 2000 ppm formaldehyde (in relation to the gelatin).

The homogenized mixtures were tempered after a reaction time of 5 min to 45° C. and mechanically foamed with air. Owing to a different relation of air to gelatin solution in the two batches, cell structures with different densities and pore sizes in accordance with Table 4 were obtained.

The casting and drying of the foamed gelatin solution were carried out as described hereinabove, similarly the cutting into discs of 2 mm thickness and the second crosslinking step under the action of formaldehyde vapor. The crosslinking time was 17 hours.

TABLE 4

| | Wet density | Dry density | Average pore |
|---|---|---|---|
| Batch | (mg/cm³) | (mg/cm³) | size (μm) |
| 4-5 | 120 | 31 | 375 |
| 4-6 | 190 | 49 | 200 |

After determining the content of excess formaldehyde in the sponges, the samples were subjected to subsequent heat treatment at 105° C. and a vacuum of approximately 14 mbar for a duration of 4 hours (sample 4-5) and 10 hours (sample 4-6), respectively. The residual content of free formaldehyde was then determined again. The results are shown in Table 5.

TABLE 5

| Batch | | |
|---|---|---|
| | 4-5 | 4-6 |

| | | | | |
|---|---|---|---|---|
| Formaldehyde content before | 0.31 | wt % | 0.33 | wt % |
| subsequent treatment | | | | |
| Duration of subsequent treatment | 4 | h | 10 | h |
| Formaldehyde content after | 0.22 | wt % | 0.20 | wt % |
| subsequent treatment | | | | |

In the case of sponge 4-5, the formaldehyde content could already be lowered by approximately 30% after 4 hours of subsequent heat treatment. In the case of the considerably denser sponge 4-6, a 10-hour subsequent treatment resulted in a lowering of the residual content by approximately 40%.

In many medical applications, a residual content of approximately 0.2 wt % represents the upper physiological limit for formaldehyde. It is apparent that in many cases this value can be achieved by the subsequent heat treatment, so that the time required for washing the sponges can be considerably shortened.

### Example 5

### Production of Shaped Bodies from Gelatin Having Undergone Prior Heat Treatment

For comparison, the pig skin gelatin (Bloom strength 300) used to produce the shaped bodies in Examples 1 to 4 was subjected to prior heat treatment as in the subsequent heat treatment of the shaped bodies in Example 4.

The gelatin was kept for 6 hours at a vacuum of approximately 14 mbar at 105° C. As a result, the Bloom strength increased from 300 to 310, the viscosity rose from 5.92 mPas to 9.04 mPas (measured in a 6.7 wt % solution at 60° C.) and the average molecular weight from 172 kDa to 189 kDa.

As in the method described in Example 1, four different films were produced from the untreated gelatin and the gelatin that had undergone prior heat treatment, respectively. The amounts for the various batches are stated in Table 6. Differently from Example 1, a 2.0 wt % aqueous formaldehyde solution was used for the first crosslinking step, and the second crosslinking step was carried out for 2 hours at the equilibrium vapor pressure of a 10% aqueous formaldehyde solution.

TABLE 6

| | Batch | | | | | | | |
|---|---|---|---|---|---|---|---|---|
| | 5-1 | | 5-2 | | 5-3 | | 5-4 | |
| Gelatin untreated | 25 | g | — | | 25 | g | — | |
| Gelatin pretreated | — | | 25 | g | — | | 25 | g |
| Water | 66 | g | 66 | g | 66 | g | 66 | g |
| Glycerin | 9 | g | 9 | g | 9 | g | 9 | g |
| Formaldehyde solution | 3.75 | g | 3.75 | g | 6.25 | g | 6.25 | g |
| Formaldehyde content in | 3000 | ppm | 3000 | ppm | 5000 | ppm | 5000 | ppm |
| relation to the gelatin | | | | | | | | |

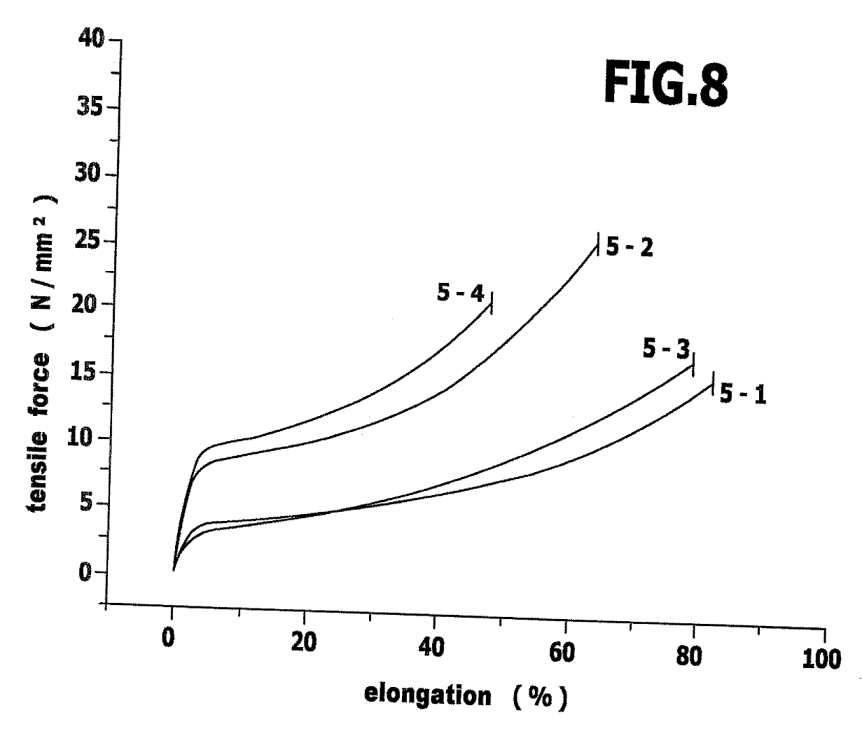

The mechanical properties of films 5-1 to 5-4 in the dry state are shown in FIG. 8. It is apparent that the tear strength of films 5-2 and 5-4 produced from the gelatin that had undergone prior heat treatment is considerably higher in comparison with the corresponding films 5-1 and 5-3 produced from the untreated gelatin. The manageability of the films in the context of medical application is thereby improved. Alternatively, it is possible to produce thinner films with comparable tear strength from the gelatin that had undergone prior heat treatment.

As far as their long-term stability under physiological conditions is concerned, the films produced from pretreated gelatin exhibit the same advantageous properties as the films made from untreated gelatin.

The gelatin that had undergone prior heat treatment is also suitable for producing shaped bodies with a cell structure as in Example 3. In this case, too, comparable long-term stabilities are observed as in the use of the untreated gelatin. Owing to the higher viscosity of the pretreated gelatin (in this case 9.04 mPas in comparison with 5.92 mPas), it is possible to significantly reduce the gelatin concentration of the solutions used for producing the sponges. Since the viscosity of a gelatin solution increases linearly to quadratically with the concentration, a 5 to 8 wt % solution of the gelatin that has undergone prior heat treatment can be used instead of a 12 wt % solution of untreated gelatin. The shaped bodies with a cell structure produced in this way are distinguished by thinner webs between the pores and a lower density, whereby, in turn, the flexibility of the sponges in increased. A lower density also means that, in total, a smaller amount of gelatin is required for use as carrier material for tissue implants.

### Example 6

### Production of Multi-Layered Sheet Materials

In accordance with the method described in Example 1, a film was produced from 33 g pig skin gelatin (Bloom strength 300), 53.25 g water, 15.5 g glycerin and 8.25 g of a 2.0 wt % formaldehyde solution, and the film applied with a doctor blade was kept for 2 hours at 40° C. prior to the drying.

A sponge of 2 to 3 mm thickness was produced in accordance with Example 3, batch 3-2.

Prior to the second crosslinking, the two sheet materials were adhesively joined to each other by means of a solution made of bone gelatin (Bloom strength 160). As described in Example 2, the multi-layered sheet material was then crosslinked by the action of formaldehyde vapor.

Alternatively to the method described here, the film and the sponge may also be each separately subjected to the second crosslinking step before the joining.

Instead of using a gelatin solution as adhesive, the two sheet materials may also be joined by the dried sponge being partially pressed into the film which has been applied with a doctor blade and has not yet been dried. A joining of the sheet materials preferably over the entire surface is obtained with both alternative procedures.

### Example 7

### Colonization of Sponges with Chondrocytes

The sponges produced in accordance with batches 3-1 to 3-3 and the reference sample crosslinked once from Example 3 were used, each as sheet material with a thickness of 2 mm, for the colonization with chondrocytes. DMEM/10% FCS/glutamine/pen/strep, which is a standard medium for cultivating mammalian cells, was used as culture medium.

Prior to the colonization, excess formaldehyde must be removed from the sponges, for example, by washing the sponges with culture medium or ethanol.

One million pig chondrocytes, suspended in 150 μ culture medium, were sown out per cm² on the sheet materials.

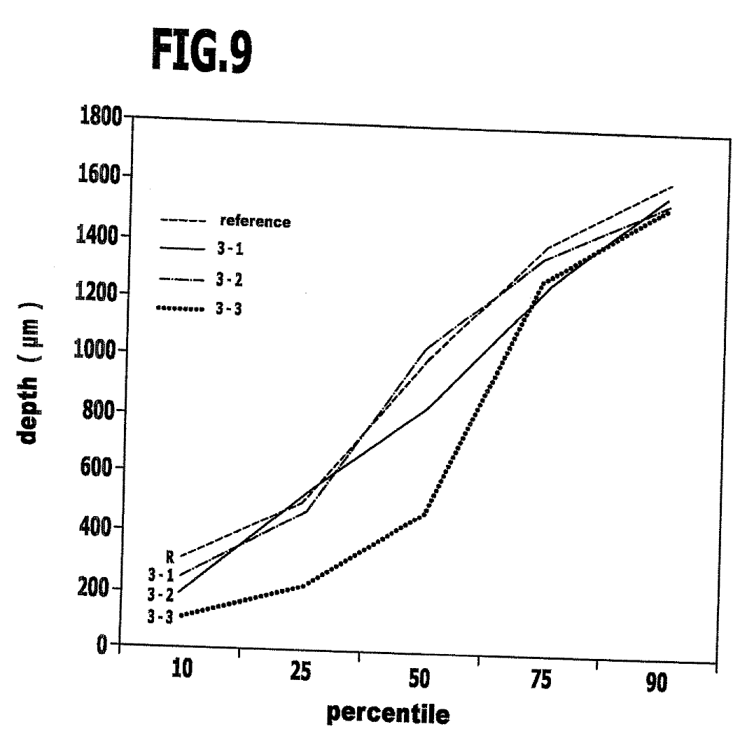

The distribution of the cells within the sponges observed after one hour is shown in FIG. 9.

The percentile indicates in percent the proportion of all cells that are distributed up to the respective colonization depth in the material. Owing to the open-pored structure, the cell distribution is substantially uniform over the entire thickness of the sponges and is not impaired by the higher degree of crosslinking of sponges 3-1 to 3-3 in comparison with reference sample R.

### Example 8

### Cultivation of Fibroblasts on Films

The films produced in accordance with batches 2-3 to 2-6 were used for the colonization with fibroblasts. DMEN/10% FCS/glutamine/pen/strep was again used as culture medium.

Prior to the colonization with cells, the films must also be washed so as to remove any residual formaldehyde.

Human foreskin fibroblasts (0.5 million cells/cm²) were sown out on the films and cultivated for 6 weeks in the medium at 37° C.

The vitality of the cells was microscopically examined twice a week. It was found that the fibroblasts on all films were viable for at least four weeks.

Furthermore, after four weeks in spite of protease production of the cells none of the films had disintegrated yet. Films 2-5 and 2-6 with a greater degree of crosslinking were even stable up until six weeks.

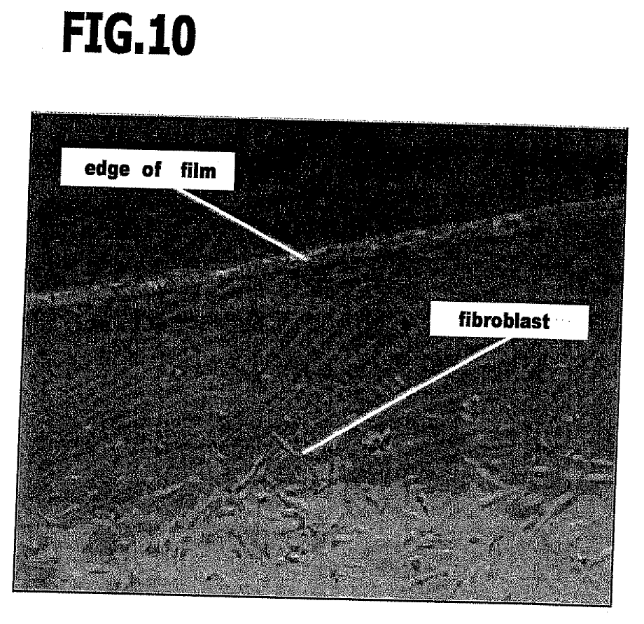

FIG. 10 shows a photomicrograph of the fibroblasts on film 2-5 after a cultivation time of 14 days. Since disintegration of the material has not yet started, the edge of the film is clearly recognizable.

Although pig skin gelatin (Bloom strength 300) was used in all of the above examples, it will be understood that comparable results are readily obtainable with other types and qualities of gelatin.

1. A method for producing shaped bodies based on crosslinked gelatin, comprising:
a) preparing an aqueous gelatin solution to provide dissolved gelatin;
b) partially crosslinking the dissolved gelatin;
c) producing a shaped body from the gelatin solution containing the partially crosslinked gelatin; and
d) crosslinking the gelatin contained in the shaped body.

2. The method in accordance with claim 1, wherein the shaped body is at least partially dried before crossing linking the gelatin contained in the shaped body

3. The method in accordance with claim 1, wherein crosslinking the gelatin contained in the shaped body is carried out by the action of a crosslinking agent in aqueous solution.

4. The method in accordance with any one of claims 1 to 3, wherein crosslinking the gelatin contained in the shaped body is carried out by the action of a crosslinking agent in the gaseous phase.

5. The method in accordance with claim 1, wherein partially crosslinking the dissolved gelatin and crosslinking the gelatin contained in the shaped body each include using a crosslinking agent, and the crosslinking agents are the same or different and are each selected from aldehydes, dialdehydes, isocyanates, diisocyanates, carbodiimides and alkyl dihalides.

6. The method in accordance with claim 5, wherein the crosslinking agent used in partially crosslinking the dissolved gelatin and/or used in crosslinking the gelatin contained in the shaped body is formaldehyde.

7. The method in accordance with claim 1, wherein excess crosslinking agent is removed from the shaped body after the crosslinking.

8. The method in accordance with claim 1, wherein the shaped body is subjected to a subsequent heat treatment at reduced pressure following crosslinking the gelatin contained in the shaped body.

9. The method in accordance with claim 8, wherein the subsequent heat treatment is carried out at a temperature of from 80 to 160° C.

10. The method in accordance with claim 1, wherein preparing the aqueous gelatin solution is carried out on the basis of a gelatin that had previously been subjected to a prior heat treatment at reduced pressure.

11. The method in accordance with claim 10, wherein the prior heat treatment is carried out at a temperature of from 80 to 160° C.

12. The method in accordance with claim 1, wherein preparing the aqueous gelatin solution is carried out on the basis of a gelatin having a viscosity of 8 mPas or more, measured in a 6.7 wt % aqueous solution at 60° C.

13. The method in accordance with claim 1, wherein the shaped body additionally contains a softener.

14. The method in accordance with claim 13, wherein the softener is selected from glycerin, oligoglycerins, oligoglycols and sorbite.

15. A shaped body, produced in accordance with the method of claim 1.

16. The shaped body in accordance with claim 15, substantially free from excess crosslinking agent.

17. The shaped body in accordance with claim 16, the shaped body having a content of excess crosslinking agent of approximately 0.2 wt % or less.

18. A shaped body based on crosslinked gelatin, the degree of crosslinking being selected such that under physiological conditions the shaped body is stable for a least one week.

19. The shaped body in accordance with claim 18, the degree of crosslinking being selected such that under physiological conditions the shaped body is stable for a least two weeks.

20. The shaped body in accordance with claim 18, the degree of crosslinking being selected such that under physiological conditions the shaped body is stable for a least four weeks.

21. The shaped body in accordance with claim 15, the shaped body being self-supporting.

22. the shaped body in accordance with claim 15, the shaped body being free from a carrier element.

23. The shaped body in accordance with claim 15, the shaped body being flexible.

24. The shaped body in accordance with claim 15, the shaped body additionally containing a softener.

25. The shaped body in accordance with claim 24, the softener being selected from glycerin, oligoglycerins, oligoglycols and sorbite.

26. The shaped body in accordance with claim 15, the shaped body being a sheet material.

27. The shaped body in accordance with claim 26, the sheet material having a cell structure.

28. The shaped body in accordance with claim 27, the cell structure being open-pored.

29. The shaped body in accordance with claim 27, wherein the cell structure is modified by mechanical action on the shaped body.

30. The shaped body in accordance with claim 29, the cell structure having cell walls, wherein some of the cell walls between the pores of the cell structure are broken.

31. The shaped body in accordance with claim 29, the density of the shaped body being increased by the mechanical action by a factor of from 2 to 10.

32. The shaped body in accordance with claim 27, wherein the pores have an average diameter of less than 300 μm.

33. The shaped body in accordance with claim 26, the sheet material being a film.

34. The shaped body in accordance with claim 33, the film having a thickness of from 20 to 500 μm.

35. A shaped body having a multilayered structure, comprising a film produced by a) preparing an aqueous gelatin solution to provide dissolved gelatin; b) partially crosslinking the dissolved gelatin; c) producing a film from the gelatin solution containing the partially crosslinked gelatin; and d) crosslinking the gelatin contained in the film; and,
a sheet material with a cell structure produced by a) preparing an aqueous gelatin solution to provide dissolved gelatin; b) partially crosslinking the dissolved gelatin; c) producing a sheet material from the gelatin solution containing the partially crosslinked gelatin and d) crosslinking the gelatin contained in the sheet material.

36. The shaped body in accordance with claim 35, the sheet material with a cell structure being directly joined to the film.

37. The shaped body in accordance with claim 36, the joining being brought about by pressing the sheet material with a cell structure into the film.

38. The shaped body in accordance with claim 35, the sheet material with a cell structure being joined to the film by means of an adhesive.

39. The shaped body in accordance with claim 38, the adhesive comprising gelatin.

40. The shaped body in accordance with claim 15, the shaped body being a hollow body.

41. The shaped body in accordance with claim 40, the hollow body being a hollow section.

42. The shaped body in accordance with claim 41, the hollow section being a hollow cylinder.

43. The shaped body in accordance with claim 40, the hollow body having a cell structure.

44. The shaped body in accordance with claim 40, the hollow body being produced by extruding a gelatin solution.

45. The shaped body in accordance with claim 41, the hollow section being produced from a sheet material prepared by a) preparing an aqueous gelatin solution to provide dissolved gelatin; b) partially crosslinking the dissolved gelatin; c) producing a sheet material from the gelatin solution containing the partially crosslinked gelatin; and d) crosslinking the gelatin contained in the sheet material.

46. A method for using a resorbable material for covering wounds or internal or external bleeding in the field of human or veterinary medicine comprising
a) preparing an aqueous gelatin solution to provide dissolved gelatin;
b) partially crosslinking the dissolved gelatin;
c) producing a resorbable material from the gelatin solution containing the partially crosslinked gelatin, including crosslinking the gelatin contained in the shaped body; and,
d) covering wounds or internal or external bleeding with the produced resorbable material.

47. A method for cultivating mammalian cells in vitro comprising
a) preparing an aqueous gelatin solution to provide dissolved gelatin;
b) partially crosslinking the dissolved gelatin;
c) producing a carrier from the gelatin solution containing the partially crosslinked gelatin; and
d) crosslinking the gelatin contained in the shaped body;
e) producing a carrier for cultivating cells;
f) colonizing the carrier with mammalian cells: and,
g) cultivating the mamalian cells.

48. The method of claim 47, wherein the mammalian cells are fibroblasts.

49. The method of claim 47, wherein the mammalian cells are chondrocytes.

50. An implant, comprising a shaped body in accordance with claim 15, and mammalian cells cultivated on the shaped body.

51. A method for treating damage, injuries and/or burns on the skin of humans or animals comprising
implanting the skin of humans or animals having damage, injuries and/or burns on the skin with the implant of claim 50.

52. A method for treating damage and/or injuries to the cartilage tissue of humans or animals comprising
implanting the implant of claim 50, into the cartilage tissue of humans or animals having damage and/or injuries to the cartilage tissue.

53. A nerve guidance channel, comprising a hollow cylinder in accordance with claim 42.

54. The nerve guidance channel in accordance with claim 53, the hollow cylinder having an inner diameter of approximately 1 mm.
